(19) United States
(12) Patent Application Publication
(10) Pub. No.: US 2019/0224261 A1
(43) Pub. Date: Jul. 25, 2019

(54) LICORICE EXTRACT COMPOSITIONS AND METHOD OF USE

(71) Applicants: Adbiotech, Co. Ltd, Chuncheon (KR); Unites Inc., Chuncheon (KR)

(72) Inventors: Jaeyoung CHO, Chuncheon (KR); Hong-Geul CHEONG, Chuncheon (KR)

(21) Appl. No.: 15/875,102

(22) Filed: Jan. 19, 2018

(51) Int. Cl. A61K 36/484; A61P 1/04

(52) U.S. Cl. CPC A61K 36/484; A61P 1/04

(57) ABSTRACT

The present disclosure provides medicinal compositions comprising licorice extracts. The disclosure also provides methods of using medicinal compositions comprising licorice extracts, in particular inflammatory-based diseases such as Crohn's Disease and ulcerative colitis.

### TECHNICAL FIELD

The invention relates to medicinal compositions comprising licorice extracts and methods of using the same. In particular, the licorice extract compositions can be used to treat gastrointestinal disorders, as well as inflammatory-based diseases such as Crohn's Disease and ulcerative colitis.

### BACKGROUND AND SUMMARY OF THE INVENTION

Gastrointestinal disorders are some of the most prevalent disease states in the world. A vast spectrum of gastrointestinal disorders is known, from disease states such as Gastroesophageal Reflux Disease (GERD), dyspepsia/indigestion and nausea and vomiting to complex disease state with inflammatory components. In particular, inflammatory-based diseases such as Crohn's Disease and ulcerative colitis are some of the most chronic and debilitating inflammatory gastrointestinal disorders.

Although many possible pharmaceutical treatments are available on the market today, many are ineffective and/or too expensive for effective treatment of affected patients. Therefore, there exists a need for new compositions and methods for improving treatment of patients inflicted with gastrointestinal disorders, as well as inflammatory-based diseases such as Crohn's Disease and ulcerative colitis. Accordingly, the present disclosure provides medicinal compositions comprising licorice extracts and methods of using the same for the treatment of various disease states.

The compositions and methods according to the present disclosure provide several advantages compared to what is known in the art. For example, the compositions of the present disclosure demonstrate improved antioxidant effects and improved anti-inflammatory effects compared to other compositions known in the art. Furthermore, the compositions of the present disclosure possess chemical properties of a particular species of licorice extracts compared to other licorice extract compositions known in the art. Moreover, the compositions of the present disclosure provide isolated extracts of licorice that contain low or miniscule levels of glycyrrhizin and higher levels of beneficial phenolic compounds than conventional licorice extracts known in the art. This feature improves the efficacy of the disclosed compounds and at the same time reduces the adverse effects compared to traditional licorice extracts.

### BRIEF DESCRIPTION OF THE DRAWINGS

FIG. 1 shows the species-specific typical constituents of G. uralensis, G. glabra, and G. inflata.

FIG. 2 shows HPLC profiles for G. uralensis, G. glabra, and G. inflata extracted with 95% ethanol compared to the comparison standard of Uralensis.

FIG. 3 shows HPLC profile differences in aqueous extraction, 50% ethanol extraction fraction, 70% ethanol extraction fraction, and 95% ethanol extraction fraction for G. inflata.

FIG. 4 shows concentrations of Licochalcone A as evaluated in aqueous extraction, 50% ethanol extraction fraction, 70% ethanol extraction fraction, and 95% ethanol extraction fraction of G. inflata.

FIG. 5A shows DSS induced symptomatic colitis parameters disease activity index (DAI) in i) mice following 10 days of oral 3% DSS administration compared to ii) control mice and to iii) mice administered the disclosed composition (“AD-lico”) at100 mg/kg. The common feature of DSS-induced colitis is an increase in the DAI. The degree of DAI was most severe between days 8 and 10 following DSS administration. Treatment with the disclosed composition prevented disease severity in mice with colitis.

FIG. 5B shows survival data of i) mice following 10 days of oral 3% DSS administration compared to ii) control mice and to iii) mice administered the disclosed composition (“AD-lico”) at100 mg/kg.

FIGS. 5C and 5D show colon length in i) mice following 10 days of oral 3% DSS administration compared to ii) control mice and to iii) mice administered the disclosed composition (“AD-lico”) at100 mg/kg. The observed decrease in colon length was significantly attenuated in 100 mg/kg AD-lico-treated mice at day 10 following DSS treatment. This finding was consistent with the observations related to weight loss and DAI index (FIG. 5A). Control: 12.28±0.23, 3% DSS: 9.50±0.48, AD-lico: 12.06±0.75

FIG. 5E shows serum C Reactive Protein (CRP) measurements in i) mice following 10 days of oral 3% DSS administration compared to ii) control mice and to iii) mice administered the disclosed composition (“AD-lico”) at100 mg/kg. Blood was collected 11 days after DSS activation by intracardiac puncture under Isoflurane anesthesia. Colonic sample were prepared as previously described (Krawisz J E et al., Gastroenterology, 1984; 84:1344-50) and CRP level was measured using enzyme-linked immunosorbent assay commercial kit (R&D Systems, Minneapolis, Minn.).

FIG. 5F shows Myeloperoxidase (MPO) measurements in i) mice following 10 days of oral 3% DSS administration compared to ii) control mice and to iii) mice administered the disclosed composition (“AD-lico”) at100 mg/kg. The mice colons (50-100 mg) were rinsed with cold PBS, blotted dry, and immediately frozen in liquid nitrogen. They were then stored at −80° C. until they were assayed for MPO activity using the o-dianisidine method. MPO activity became markedly increased, to a level approximately 3.3 times higher than that in the control group. This increase in MPO activity was significantly reduced by AD-lico administration. AD-lico administration (100 mg/kg) suppressed the MPO activity to basal level. MPO is an enzyme produced mainly by polymorphonuclear leucocytes and associated with the degree of neutrophil infiltration in tissues and MPO activity is considered a biochemical marker of neutrophil infiltration. This result demonstrates that AD-lico exerts anti-inflammatory effects by reducing neutrophil infiltration into the colonic mucosa.

FIG. 6 shows an improvement in dose dependent mucosal damage caused by H. pylori in rats. Column A: normal control; Column B: negative control; Column C: AD-lico, 25 mg/kg; Column D: AD-lico, 25 mg/kg; Column E: AD-lico, 100 mg/kg. The pathologic scores from the tissue slides were calculated and plotted.

FIG. 7 shows a Western blot of treated cell lysates were probed for iNOS, COX-2, and actin. Lane A: normal control; Lane B: negative control; Lane C: AD-lico, 25 mg/kg; Lane D: AD-lico, 50 mg/kg; Lane E: AD-lico, 100 mg/kg.

FIGS. 8A, 8B, 8C, and 8D show an evaluation of mucosal damage caused by water immersion stress in rat stomachs in one of four groups: FIG. 8A shows normal control; FIG. 8B shows negative control; FIG. 8C shows AD-lico, 25 mg/kg; and FIG. 8D shows SWG, an approved functional food in Korea, 50 mg/kg.

FIG. 9A shows photographs of the stomachs of sacrificed rats in one of four groups: G1 (normal control); G2 (negative control); G3 (mosapride, 10 mg/kg) and G4 (AD-lico, 100 mg/kg).

FIG. 9B shows quantitated gastric emptying rates (%) of sacrificed rats in one of four groups: G1 (normal control); G2 (negative control); G3 (mosapride, 10 mg/kg) and G4 (AD-lico, 100 mg/kg).

### DETAILED DESCRIPTION

Various embodiments of the invention are described herein as follows. In one aspect, a medicinal composition comprising one or more extract components of a licorice is provided. In another aspect, a method of treating a gastrointestinal disorder is provided. The method comprises a step of administering a medicinal composition comprising one or more extract components of a licorice to a patient in need thereof. In yet another aspect, a method of treating an inflammatory disease is provided. The method comprises a step of administering a medicinal composition comprising one or more extract components of a licorice to a patient in need thereof.

The following numbered embodiments are contemplated and are non-limiting:

1. A medicinal composition comprising one or more extract components of a licorice.

2. The medicinal composition of clause 1, wherein the licorice is Glycyrrhiza inflata batal.

3. The medicinal composition of clause 1 or clause 2, wherein the extract component is Licochalcone A.

4. The medicinal composition of clause 3, wherein the Licochalcone A is present between about 0.5% and about 6%.

5. The medicinal composition of clause 3, wherein the Licochalcone A is present between about 1% and about 6%.

6. The medicinal composition of clause 3, wherein the Licochalcone A is present between about 1.5% and about 6%.

7. The medicinal composition of clause 3, wherein the Licochalcone A is present between about 2% and about 6%.

8. The medicinal composition of clause 3, wherein the Licochalcone A is present between about 2.5% and about 6%.

9. The medicinal composition of clause 3, wherein the Licochalcone A is present between about 3% and about 6%.

10. The medicinal composition of clause 3, wherein the Licochalcone A is present between about 0.5% and about 5%.

11. The medicinal composition of clause 3, wherein the Licochalcone A is present between about 0.5% and about 4.5%.

12. The medicinal composition of clause 3, wherein the Licochalcone A is present between about 0.5% and about 4%.

13. The medicinal composition of clause 3, wherein the Licochalcone A is present between about 0.5% and about 3.5%.

14. The medicinal composition of clause 3, wherein the Licochalcone A is present between about 0.5% and about 3%.

15. The medicinal composition of clause 3, wherein the Licochalcone A is present at about 0.5%.

16. The medicinal composition of clause 3, wherein the Licochalcone A is present at about 1%.

17. The medicinal composition of clause 3, wherein the Licochalcone A is present at about 2%.

18. The medicinal composition of clause 3, wherein the Licochalcone A is present at about 3%.

19. The medicinal composition of clause 3, wherein the Licochalcone A is present at about 4%.

20. The medicinal composition of clause 3, wherein the Licochalcone A is present at about 5%.

21. The medicinal composition of clause 3, wherein the Licochalcone A is present at about 6%.

22. The medicinal composition of any one of clauses 1 to 21, wherein the composition is substantially free of glycyrrhizin.

23. The medicinal composition of any one of clauses 1 to 21, wherein the composition contains less than about 3% glycyrrhizin.

24. The medicinal composition of any one of clauses 1 to 21, wherein the composition contains less than about 2.5% glycyrrhizin.

25. The medicinal composition of any one of clauses 1 to 21, wherein the composition contains less than about 2% glycyrrhizin.

26. The medicinal composition of any one of clauses 1 to 21, wherein the composition contains less than about 1.5% glycyrrhizin.

27. The medicinal composition of any one of clauses 1 to 21, wherein the composition contains less than about 1% glycyrrhizin.

28. The medicinal composition of any one of clauses 1 to 21, wherein the composition contains less than about 0.5% glycyrrhizin.

29. The medicinal composition of any one of clauses 1 to 28, wherein the extract component is echinatin.

30. The medicinal composition of clause 29, wherein the echinatin is present between about 0.1% and about 0.5%.

31. The medicinal composition of clause 29, wherein the echinatin is present between about 0.1% and about 0.4%.

32. The medicinal composition of clause 29, wherein the echinatin is present between about 0.1% and about 0.3%.

33. The medicinal composition of clause 29, wherein the echinatin is present between about 0.1% and about 0.2%.

34. The medicinal composition of clause 29, wherein the echinatin is present between about 0.2% and about 0.5%.

35. The medicinal composition of clause 29, wherein the echinatin is present between about 0.3% and about 0.5%.

36. The medicinal composition of clause 29, wherein the echinatin is present between about 0.4% and about 0.5%.

37. The medicinal composition of clause 29, wherein the echinatin is present at about 0.1%.

38. The medicinal composition of clause 29, wherein the echinatin is present at about 0.2%.

39. The medicinal composition of clause 29, wherein the echinatin is present at about 0.3%.

40. The medicinal composition of clause 29, wherein the echinatin is present at about 0.4%.

41. The medicinal composition of clause 29, wherein the echinatin is present at about 0.5%.

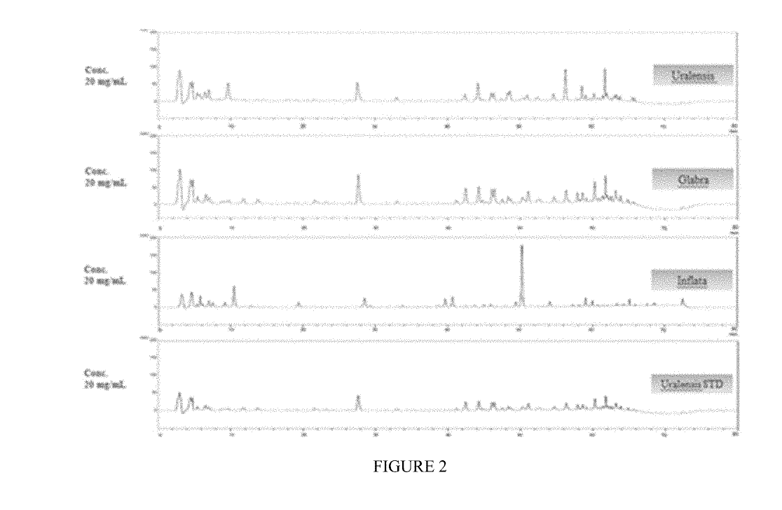

42. The medicinal composition of any one of clauses 1 to 42, wherein wherein the composition has the HPLC profile of FIG. 2.

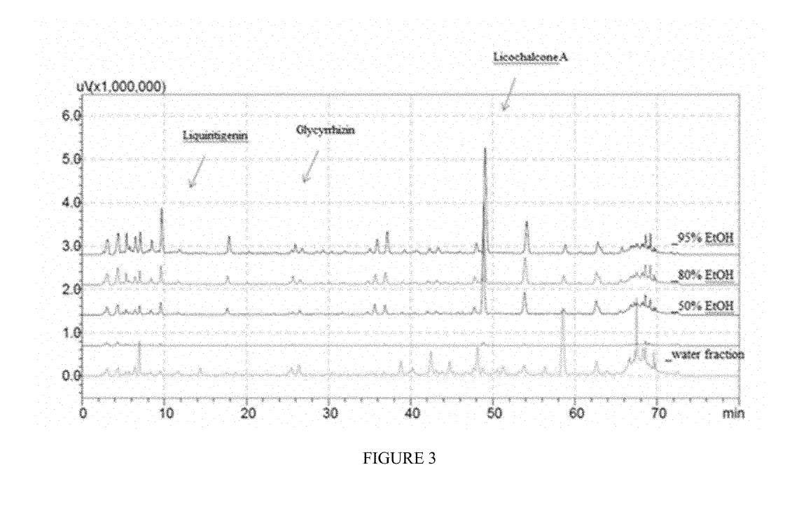

43. The medicinal composition of any one of clauses 1 to 42, wherein the composition has the HPLC profile of FIG. 3.

44. The medicinal composition of any one of clauses 1 to 43, wherein the extract is an ethanol extract.

45. The medicinal composition of clause 44, wherein the ethanol extract is between 50% and 95% ethanol extract.

46. The medicinal composition of clause 44, wherein the ethanol extract is a 50% ethanol extract.

47. The medicinal composition of clause 44, wherein the ethanol extract is a 70% ethanol extract.

48. The medicinal composition of clause 44, wherein the ethanol extract is a 75% ethanol extract.

49. The medicinal composition of clause 44, wherein the ethanol extract is a 80% ethanol extract.

50. The medicinal composition of clause 44, wherein the ethanol extract is a 90% ethanol extract.

51. The medicinal composition of clause 44, wherein the ethanol extract is a 95% ethanol extract.

52. The medicinal composition of any one of clauses 1 to 51, wherein the composition comprises a mixture of extract components selected from the group consisting of Licochalcone A and glycyrrhizin.

53. The medicinal composition of clause 52, wherein the extract components are present at between about 0.5% and about 6% of Licochalcone A and less than about 3% glycyrrhizin.

54. The medicinal composition of clause 52 or clause 53, wherein the Licochalcone A is present between about 0.5% and about 6%.

55. The medicinal composition of clause 52 or clause 53, wherein the Licochalcone A is present between about 1% and about 6%.

56. The medicinal composition of clause 52 or clause 53, wherein the Licochalcone A is present between about 1.5% and about 6%.

57. The medicinal composition of clause 52 or clause 53, wherein the Licochalcone A is present between about 2% and about 6%.

58. The medicinal composition of clause 52 or clause 53, wherein the Licochalcone A is present between about 2.5% and about 6%.

59. The medicinal composition of clause 52 or clause 53, wherein the Licochalcone A is present between about 3% and about 6%.

60. The medicinal composition of clause 52 or clause 53, wherein the Licochalcone A is present between about 0.5% and about 5%.

61. The medicinal composition of clause 52 or clause 53, wherein the Licochalcone A is present between about 0.5% and about 4.5%.

62. The medicinal composition of clause 52 or clause 53, wherein the Licochalcone A is present between about 0.5% and about 4%.

63. The medicinal composition of clause 52 or clause 53, wherein the Licochalcone A is present between about 0.5% and about 3.5%.

64. The medicinal composition of clause 52 or clause 53, wherein the Licochalcone A is present between about 0.5% and about 3%.

65. The medicinal composition of clause 52 or clause 53, wherein the Licochalcone A is present at about 0.5%.

66. The medicinal composition of clause 52 or clause 53, wherein the Licochalcone A is present at about 1%.

67. The medicinal composition of clause 52 or clause 53, wherein the Licochalcone A is present at about 2%.

68. The medicinal composition of clause 52 or clause 53, wherein the Licochalcone A is present at about 3%.

69. The medicinal composition of clause 52 or clause 53, wherein the Licochalcone A is present at about 4%.

70. The medicinal composition of clause 52 or clause 53, wherein the Licochalcone A is present at about 5%.

71. The medicinal composition of clause 52 or clause 53, wherein the Licochalcone A is present at about 6%. 72. The medicinal composition of any one of clauses 52 to 71, wherein the composition contains less than about 3% glycyrrhizin.

73. The medicinal composition of any one of clauses 52 to 71, wherein the composition contains less than about 2.5% glycyrrhizin.

74. The medicinal composition of any one of clauses 52 to 71, wherein the composition contains less than about 2% glycyrrhizin.

75. The medicinal composition of any one of clauses 52 to 71, wherein the composition contains less than about 1.5% glycyrrhizin.

76. The medicinal composition of any one of clauses 52 to 71, wherein the composition contains less than about 1% glycyrrhizin.

77. The medicinal composition of any one of clauses 52 to 71, wherein the composition contains less than about 0.5% glycyrrhizin.

78. The medicinal composition of any one of clauses 1 to 51, wherein the composition comprises a mixture of extract components selected from the group consisting of Licochalcone A and echinatin.

79. The medicinal composition of clause 78, wherein the extract components are present at between about 0.5% and about 6% of Licochalcone A and between about 0.1% and about 0.5% of echinatin.

80. The medicinal composition of clause 78 or clause 79, wherein the Licochalcone A is present between about 0.5% and about 6%.

81. The medicinal composition of clause 78 or clause 79, wherein the Licochalcone A is present between about 1% and about 6%.

82. The medicinal composition of clause 78 or clause 79, wherein the Licochalcone A is present between about 1.5% and about 6%.

83. The medicinal composition of clause 78 or clause 79, wherein the Licochalcone A is present between about 2% and about 6%.

84. The medicinal composition of clause 78 or clause 79, wherein the Licochalcone A is present between about 2.5% and about 6%.

85. The medicinal composition of clause 78 or clause 79, wherein the Licochalcone A is present between about 3% and about 6%.

86. The medicinal composition of clause 78 or clause 79, wherein the Licochalcone A is present between about 0.5% and about 5%.

87. The medicinal composition of clause 78 or clause 79, wherein the Licochalcone A is present between about 0.5% and about 4.5%.

88. The medicinal composition of clause 78 or clause 79, wherein the Licochalcone A is present between about 0.5% and about 4%.

89. The medicinal composition of clause 78 or clause 79, wherein the Licochalcone A is present between about 0.5% and about 3.5%.

90. The medicinal composition of clause 78 or clause 79, wherein the Licochalcone A is present between about 0.5% and about 3%.

91. The medicinal composition of clause 78 or clause 79, wherein the Licochalcone A is present at about 0.5%.

92. The medicinal composition of clause 78 or clause 79, wherein the Licochalcone A is present at about 1%.

93. The medicinal composition of clause 78 or clause 79, wherein the Licochalcone A is present at about 2%.

94. The medicinal composition of clause 78 or clause 79, wherein the Licochalcone A is present at about 3%.

95. The medicinal composition of clause 78 or clause 79, wherein the Licochalcone A is present at about 4%.

96. The medicinal composition of clause 78 or clause 79, wherein the Licochalcone A is present at about 5%.

97. The medicinal composition of clause 78 or clause 79, wherein the Licochalcone A is present at about 6%.

98. The medicinal composition of any one of clauses 78 to 97, wherein the echinatin is present between about 0.1% and about 0.5%.

99. The medicinal composition of any one of clauses 78 to 97, wherein the echinatin is present between about 0.1% and about 0.4%.

100. The medicinal composition of any one of clauses 78 to 97, wherein the echinatin is present between about 0.1% and about 0.3%.

101. The medicinal composition of any one of clauses 78 to 97, wherein the echinatin is present between about 0.1% and about 0.2%.

102. The medicinal composition of any one of clauses 78 to 97, wherein the echinatin is present between about 0.2% and about 0.5%.

103. The medicinal composition of any one of clauses 78 to 97, wherein the echinatin is present between about 0.3% and about 0.5%.

104. The medicinal composition of any one of clauses 78 to 97, wherein the echinatin is present between about 0.4% and about 0.5%.

105. The medicinal composition of any one of clauses 78 to 97, wherein the echinatin is present at about 0.1%.

106. The medicinal composition of any one of clauses 78 to 97, wherein the echinatin is present at about 0.2%.

107. The medicinal composition of any one of clauses 78 to 97, wherein the echinatin is present at about 0.3%.

108. The medicinal composition of any one of clauses 78 to 97, wherein the echinatin is present at about 0.4%.

109. The medicinal composition of any one of clauses 78 to 97, wherein the echinatin is present at about 0.5%.

110. The medicinal composition of any one of clauses 1 to 51, wherein the composition comprises a mixture of extract components selected from the group consisting of Licochalcone A, glycyrrhizin, and echinatin.

111. The medicinal composition of clause 110, wherein the extract components are present at between about 0.5% and about 6% of Licochalcone A, less than about 3% glycyrrhizin, and between about 0.1% and about 0.5% of echinatin.

112. The medicinal composition of clause 110 or clause 111, wherein the Licochalcone A is present between about 0.5% and about 6%.

113. The medicinal composition of clause 110 or clause 111, wherein the Licochalcone A is present between about 1% and about 6%.

114. The medicinal composition of clause 110 or clause 111, wherein the Licochalcone A is present between about 1.5% and about 6%.

115. The medicinal composition of clause 110 or clause 111, wherein the Licochalcone A is present between about 2% and about 6%.

116. The medicinal composition of clause 110 or clause 111, wherein the Licochalcone A is present between about 2.5% and about 6%.

117. The medicinal composition of clause 110 or clause 111, wherein the Licochalcone A is present between about 3% and about 6%.

118. The medicinal composition of clause 110 or clause 111, wherein the Licochalcone A is present between about 0.5% and about 5%.

119. The medicinal composition of clause 110 or clause 111, wherein the Licochalcone A is present between about 0.5% and about 4.5%.

120. The medicinal composition of clause 110 or clause 111, wherein the Licochalcone A is present between about 0.5% and about 4%.

121. The medicinal composition of clause 110 or clause 111, wherein the Licochalcone A is present between about 0.5% and about 3.5%.

122. The medicinal composition of clause 110 or clause 111, wherein the Licochalcone A is present between about 0.5% and about 3%.

123. The medicinal composition of clause 110 or clause 111, wherein the Licochalcone A is present at about 0.5%.

124. The medicinal composition of clause 110 or clause 111, wherein the Licochalcone A is present at about 1%.

125. The medicinal composition of clause 110 or clause 111, wherein the Licochalcone A is present at about 2%.

126. The medicinal composition of clause 110 or clause 111, wherein the Licochalcone A is present at about 3%.

127. The medicinal composition of clause 110 or clause 111, wherein the Licochalcone A is present at about 4%.

128. The medicinal composition of clause 110 or clause 111, wherein the Licochalcone A is present at about 5%.

129. The medicinal composition of clause 110 or clause 111, wherein the Licochalcone A is present at about 6%.

130. The medicinal composition of any one of clauses 110 to 129, wherein the composition contains less than about 3% glycyrrhizin.

131. The medicinal composition of any one of clauses 110 to 129, wherein the composition contains less than about 2.5% glycyrrhizin.

132. The medicinal composition of any one of clauses 110 to 129, wherein the composition contains less than about 2% glycyrrhizin.

133. The medicinal composition of any one of clauses 110 to 129, wherein the composition contains less than about 1.5% glycyrrhizin.

134. The medicinal composition of any one of clauses 110 to 129, wherein the composition contains less than about 1% glycyrrhizin.

135. The medicinal composition of any one of clauses 110 to 129, wherein the composition contains less than about 0.5% glycyrrhizin.

136. The medicinal composition of any one of clauses 110 to 135, wherein the echinatin is present between about 0.1% and about 0.5%.

137. The medicinal composition of any one of clauses 110 to 135, wherein the echinatin is present between about 0.1% and about 0.4%.

138. The medicinal composition of any one of clauses 110 to 135, wherein the echinatin is present between about 0.1% and about 0.3%.

139. The medicinal composition of any one of clauses 110 to 135, wherein the echinatin is present between about 0.1% and about 0.2%.

140. The medicinal composition of any one of clauses 110 to 135, wherein the echinatin is present between about 0.2% and about 0.5%.

141. The medicinal composition of any one of clauses 110 to 135, wherein the echinatin is present between about 0.3% and about 0.5%.

142. The medicinal composition of any one of clauses 110 to 135, wherein the echinatin is present between about 0.4% and about 0.5%.

143. The medicinal composition of any one of clauses 110 to 135, wherein the echinatin is present at about 0.1%.

144. The medicinal composition of any one of clauses 110 to 135, wherein the echinatin is present at about 0.2%.

145. The medicinal composition of any one of clauses 110 to 135, wherein the echinatin is present at about 0.3%.

146. The medicinal composition of any one of clauses 110 to 135, wherein the echinatin is present at about 0.4%.

147. The medicinal composition of any one of clauses 110 to 135, wherein the echinatin is present at about 0.5%.

148. A method of treating a gastrointestinal disorder, said method comprising a step of administering a medicinal composition comprising one or more extract components of a licorice to a patient in need thereof.

149. The method of clause 148, wherein the medicinal composition is a medicinal composition of any one of clauses 1 to 147.

150. The method of clause 148 or clause 149, wherein the gastrointestinal disorder is gastritis.

151. The method of clause 148 or clause 149, wherein the gastrointestinal disorder is an ulcer.

152. The method of clause 148 or clause 149, wherein the gastrointestinal disorder is reflux disease.

153. The method of clause 148 or clause 149, wherein the gastrointestinal disorder is erosion.

154. The method of clause 148 or clause 149, wherein the gastrointestinal disorder is gastric discomfort.

155. The method of clause 148 or clause 149, wherein the gastrointestinal disorder is gastric immotility.

156. The method of any one of clauses 148 to 155, wherein the method reduces interleukin-6 (IL-6) concentration in the patient.

157. The method of any one of clauses 148 to 156, wherein the method reduces nitrous oxide (NO) concentration in the patient.

158. The method of any one of clauses 148 to 157, wherein the method reduces cyclooxygenase-2 (COX-2) concentration in the patient.

159. The method of any one of clauses 148 to 158, wherein the method reduces tumor necrosis factor-α (TNF-α) concentration in the patient.

160. The method of any one of clauses 148 to 159, wherein the method reduces C-reactive protein (CRP) concentration in the patient.

161. The method of any one of clauses 148 to 160, wherein the medicinal composition is administered to the patient between 100 mg/day and 500 mg/day.

162. The method of any one of clauses 148 to 160, wherein the medicinal composition is administered to the patient between 100 mg/day and 400 mg/day.

163. The method of any one of clauses 148 to 160, wherein the medicinal composition is administered to the patient between 250 mg/day and 500 mg/day.

164. The method of any one of clauses 148 to 160, wherein the medicinal composition is administered to the patient at 100 mg/day.

165. The method of any one of clauses 148 to 160, wherein the medicinal composition is administered to the patient at 150 mg/day.

166. The method of any one of clauses 148 to 160, wherein the medicinal composition is administered to the patient at 200 mg/day.

167. The method of any one of clauses 148 to 160, wherein the medicinal composition is administered to the patient at 250 mg/day.

168. The method of any one of clauses 148 to 160, wherein the medicinal composition is administered to the patient at 300 mg/day.

169. The method of any one of clauses 148 to 160, wherein the medicinal composition is administered to the patient at 350 mg/day.

170. The method of any one of clauses 148 to 160, wherein the medicinal composition is administered to the patient at 400 mg/day.

171. The method of any one of clauses 148 to 160, wherein the medicinal composition is administered to the patient between at 450 mg/day.

172. The method of any one of clauses 148 to 160, wherein the medicinal composition is administered to the patient at 500 mg/day.

173. A method of treating an inflammatory disease, said method comprising a step of administering a medicinal composition comprising one or more extract components of a licorice to a patient in need thereof.

174. The method of clause 173, wherein the medicinal composition is a medicinal composition of any one of clauses 1 to 147.

175. The method of clause 173 or clause 174, wherein the inflammatory disease is an inflammatory bowel disease.

176. The method of clause 173 or clause 174, wherein the inflammatory disease is ulcerative colitis.

177. The method of clause 173 or clause 174, wherein the inflammatory disease is Crohn's disease.

178. The method of any one of clauses 173 to 177, wherein the method reduces interleukin-6 (IL-6) concentration in the patient.

179. The method of any one of clauses 173 to 178, wherein the method reduces nitrous oxide (NO) concentration in the patient.

180. The method of any one of clauses 173 to 179, wherein the method reduces cyclooxygenase-2 (COX-2) concentration in the patient.

181. The method of any one of clauses 173 to 180, wherein the method reduces tumor necrosis factor-α (TNF-α) concentration in the patient.

182. The method of any one of clauses 173 to 181, wherein the method reduces C-reactive protein (CRP) concentration in the patient.

183. The method of any one of clauses 173 to 182, wherein the medicinal composition is administered to the patient between 100 mg/day and 500 mg/day.

184. The method of any one of clauses 173 to 182, wherein the medicinal composition is administered to the patient between 100 mg/day and 400 mg/day.

185. The method of any one of clauses 173 to 182, wherein the medicinal composition is administered to the patient between 250 mg/day and 500 mg/day.

186. The method of any one of clauses 173 to 182, wherein the medicinal composition is administered to the patient at 100 mg/day.

187. The method of any one of clauses 173 to 182, wherein the medicinal composition is administered to the patient at 150 mg/day.

188. The method of any one of clauses 173 to 182, wherein the medicinal composition is administered to the patient at 200 mg/day.

189. The method of any one of clauses 173 to 182, wherein the medicinal composition is administered to the patient at 250 mg/day.

190. The method of any one of clauses 173 to 182, wherein the medicinal composition is administered to the patient at 300 mg/day.

191. The method of any one of clauses 173 to 182, wherein the medicinal composition is administered to the patient at 350 mg/day.

192. The method of any one of clauses 173 to 182, wherein the medicinal composition is administered to the patient at 400 mg/day.

193. The method of any one of clauses 173 to 182, wherein the medicinal composition is administered to the patient between at 450 mg/day.

194. The method of any one of clauses 173 to 182, wherein the medicinal composition is administered to the patient at 500 mg/day.

195. A method of promoting gastric motility of a patient, said method comprising a step of administering a medicinal composition comprising one or more extract components of a licorice to the patient.

196. The method of clause 195, wherein the medicinal composition is a medicinal composition of any one of clauses 1 to 147.

197. The method of clause 195 or clause 196, wherein the method accelerates gastric emptying.

198. The method of clause 195 or clause 196, wherein the method promotes gastric relaxation.

199. The method of any one of clauses 195 to 198, wherein the method reduces interleukin-6 (IL-6) concentration in the patient.

200. The method of any one of clauses 195 to 199, wherein the method reduces nitrous oxide (NO) concentration in the patient.

201. The method of any one of clauses 195 to 200, wherein the method reduces cyclooxygenase-2 (COX-2) concentration in the patient.

202. The method of any one of clauses 195 to 201, wherein the method reduces tumor necrosis factor-α (TNF-α) concentration in the patient.

203. The method of any one of clauses 195 to 202, wherein the method reduces C-reactive protein (CRP) concentration in the patient.

204. The method of any one of clauses 195 to 203, wherein the medicinal composition is administered to the patient between 100 mg/day and 500 mg/day.

205. The method of any one of clauses 195 to 203, wherein the medicinal composition is administered to the patient between 100 mg/day and 400 mg/day.

206. The method of any one of clauses 195 to 203, wherein the medicinal composition is administered to the patient between 250 mg/day and 500 mg/day.

207. The method of any one of clauses 195 to 203, wherein the medicinal composition is administered to the patient at 100 mg/day.

208. The method of any one of clauses 195 to 203, wherein the medicinal composition is administered to the patient at 150 mg/day.

209. The method of any one of clauses 195 to 203, wherein the medicinal composition is administered to the patient at 200 mg/day.

210. The method of any one of clauses 195 to 203, wherein the medicinal composition is administered to the patient at 250 mg/day.

211. The method of any one of clauses 195 to 203, wherein the medicinal composition is administered to the patient at 300 mg/day.

212. The method of any one of clauses 195 to 203, wherein the medicinal composition is administered to the patient at 350 mg/day.

213. The method of any one of clauses 195 to 203, wherein the medicinal composition is administered to the patient at 400 mg/day.

214. The method of any one of clauses 195 to 203, wherein the medicinal composition is administered to the patient between at 450 mg/day.

215. The method of any one of clauses 195 to 203, wherein the medicinal composition is administered to the patient at 500 mg/day.

216. A method of reducing mucosal damage in a patient, said method comprising a step of administering a medicinal composition comprising one or more extract components of a licorice to the patient.

217. The method of clause 216, wherein the medicinal composition is a medicinal composition of any one of clauses 1 to 147.

218. The method of clause 216 or clause 217, wherein the mucosal damage is caused by inflammation.

219. The method of any one of clauses 216 to 218, wherein the method reduces interleukin-6 (IL-6) concentration in the patient.

220. The method of any one of clauses 216 to 219, wherein the method reduces nitrous oxide (NO) concentration in the patient.

221. The method of any one of clauses 216 to 220, wherein the method reduces cyclooxygenase-2 (COX-2) concentration in the patient.

222. The method of any one of clauses 216 to 221, wherein the method reduces tumor necrosis factor-α (TNF-α) concentration in the patient.

223. The method of any one of clauses 216 to 222, wherein the method reduces C-reactive protein (CRP) concentration in the patient.

224. The method of any one of clauses 216 to 223, wherein the medicinal composition is administered to the patient between 100 mg/day and 500 mg/day.

225. The method of any one of clauses 216 to 223, wherein the medicinal composition is administered to the patient between 100 mg/day and 400 mg/day.

226. The method of any one of clauses 216 to 223, wherein the medicinal composition is administered to the patient between 250 mg/day and 500 mg/day.

227. The method of any one of clauses 216 to 223, wherein the medicinal composition is administered to the patient at 100 mg/day.

228. The method of any one of clauses 216 to 223, wherein the medicinal composition is administered to the patient at 150 mg/day.

229. The method of any one of clauses 216 to 223, wherein the medicinal composition is administered to the patient at 200 mg/day.

230. The method of any one of clauses 216 to 223, wherein the medicinal composition is administered to the patient at 250 mg/day.

231. The method of any one of clauses 216 to 223, wherein the medicinal composition is administered to the patient at 300 mg/day.

232. The method of any one of clauses 216 to 223, wherein the medicinal composition is administered to the patient at 350 mg/day.

233. The method of any one of clauses 216 to 223, wherein the medicinal composition is administered to the patient at 400 mg/day.

234. The method of any one of clauses 216 to 223, wherein the medicinal composition is administered to the patient between at 450 mg/day.

235. The method of any one of clauses 216 to 223, wherein the medicinal composition is administered to the patient at 500 mg/day.

In one aspect, a medicinal composition comprising one or more extract components of a licorice is provided. As used herein, extract components of a licorice refer to any compound or combination of compounds in the glycyrrhiza family, derivatives thereof, and combinations thereof. Specific examples of derivatives of licorice extracts include salts (e.g., metal salts, ammonium salts, and the like) and esters (e.g. saturated fatty acid esters, unsaturated fatty acid esters, diacid half esters, glycoside esters, and the like). In certain embodiments, the licorice is Glycyrrhiza inflata batal.

In some embodiments, the extract component is Licochalcone A. Licochalcone A is a chalconoid and may be isolated, for example, from the root of Glycyrrhiza glabra or Glycyrrhiza inflata. In various embodiments, the composition is substantially free of glycyrrhizin. Glycyrrhizin (aka glycyrrhizic acid or glycyrrhizinic acid) is the chief sweet-tasting constituent of a licorice root (e.g., a Glycyrrhiza glabra root). As used herein, the term “substantially free” refers to a non-appreciable amount of glycyrrhizin that may be present in a medicinal composition of the present invention. In some embodiments, the extract component is echinatin.

In some embodiments, the composition comprises a mixture of extract components selected from the group consisting of Licochalcone A and glycyrrhizin. In other embodiments, the composition comprises a mixture of extract components selected from the group consisting of Licochalcone A and echinatin. In yet other embodiments, the composition comprises a mixture of extract components selected from the group consisting of Licochalcone A, glycyrrhizin, and echinatin.

In another aspect, a method of treating a gastrointestinal disorder is provided. The method comprises a step of administering a medicinal composition comprising one or more extract components of a licorice to a patient in need thereof. The previously described embodiments of the medicinal composition are applicable to the method of treating a gastrointestinal disorder described herein. As used herein, “gastrointestinal disorder” refers to any disorder of the stomach, small intestine and/or large intestine. As used herein, “administer”, “administering,” or “administered” is an oral administration that refers to the provision of a medicinal composition via the mouth through ingestion, or via some other part of the gastrointestinal system including the esophagus. Examples of oral dosage forms include tablets (including compressed, coated or uncoated), capsules, hard or soft gelatin capsules, pellets, pills, powders, granules, elixirs, tinctures, colloidal dispersions, dispersions, effervescent compositions, films, sterile solutions or suspensions, syrups and emulsions and the like.

In yet another aspect, a method of treating an inflammatory disease is provided. The method comprises a step of administering a medicinal composition comprising one or more extract components of a licorice to a patient in need thereof. The previously described embodiments of the medicinal composition and of the method of treating a gastrointestinal disorder are applicable to the method of treating an inflammatory disease described herein. As used herein, “inflammatory disease” refers to a disease or condition involving an inflammatory response. The inflammatory response may be acute and/or chronic.

In another aspect, a method of promoting gastric motility is provided. The method comprises a step of administering a medicinal composition comprising one or more extract components of a licorice to a patient in need thereof. The previously described embodiments of the medicinal composition, of the method of treating a gastrointestinal disorder, and of the method of treating an inflammatory disease are applicable to the method of promoting gastric motility described herein. As used herein, “promoting gastric motility” refers to promoting the contractility of the muscle of the esophagus, the small intestine, the large intestine, and/or muscle tissue there between.

In yet another aspect, a method of reducing mucosal damage is provided. The method comprises a step of administering a medicinal composition comprising one or more extract components of a licorice to a patient in need thereof. The previously described embodiments of the medicinal composition, of the method of treating a gastrointestinal disorder, of the method of treating an inflammatory disease, and of method of promoting gastric motility are applicable to the method of reducing mucosal damage described herein. As used herein, “mucosal damage” includes, but is not limited to damage caused by bacterial, viral, and fungal infections, ulcerations, autoimmune disorders, septic shock, allergic and non-allergic rhinitis, hemorrhagic shock, endotoxemia, oral mucositis, burning mouth syndrome, lichen planus, denture sores, gingivitus, recent oral surgical sites, cervical dysplasia, vulva leukoplakia and other vulval lesions, Bechets Syndrome, radiotherapy induced mucositis, post-operative gum pain, traumatic mouth lesions, post-radiotherapy vaginitis, non-specific vaginal inflammatory conditions, and other viral auto-immune and inflammatory ulcerations of the mucosa, nonspecific ulcer of colon, ulcerative colitis induced by nonspecific inflammations, and Crohn's disease.

While the invention is susceptible to various modifications and alternative forms, specific embodiments are herein described in detail. It should be understood, however, that there is no intent to limit the invention to the particular forms described, but on the contrary, the intention is to cover all modifications, equivalents, and alternatives falling within the scope of the invention.

### Example 1

### Preparation of Composition Comprising a Licorice Extract

The preparation processes for the medicinal composition comprising one or more extract components of a licorice involves steps for licorice root powder roasting, ethanol extraction, concentration, precipitation, washing of the extract powder and drying the extract powder all under controlled conditions and with quality control. An exemplary process to prepare the compositions is as follows.

The stem and roots of the G. inflata plant were cut and baked for approximately 30 minutes at between 150-180° C. The baking process may be done by Humanherb. Thereafter, the dried product is combined with ethanol at approximately a 1 kg dried product: 10 L ethanol ratio. For example, 700 kg of the dried product may be combined with about 7000 L of 95% ethanol, or 500 kg of the dried product may be combined with about 5000 L of 95% ethanol.

A reflux extraction method is then performed on the combined product at about 70-80° C. for at least 10 hours. Thereafter, a vacuum filter was applied to the extract (e.g., a cartridge filter of φ 10 μm. Vacuum evaporation is performed at <60° C., <600 mmHG, and <2 kgf/cm² until 100 L of extract is achieved.

Thereafter, purified water is added to the extract at approximately two times (2×) the volume of the extract. The mixture is stirred for 1 hour and stored for at least 10 hours. Thereafter, the precipitate is collected.

The precipitate is washed with purified water (at approximately two times (2×) the volume of the extract), stirred for 3 hours, and a Nutsche filter is applied (using polyester 100 Mesh). This washing step is then repeated one or two times.

Thereafter, the product is obtained via freeze drying (lyophilization) at −60° C. For instance, the filtered precipitate can be freezed at −70° C. for 72 hours and freeze drying can occur for 72 hours at −60° C.

The product is then grinded at 1500 RPM using a filter with 50 Mesh. Finally, the ground product can be packaged with PE vinyl, then with 5 mm thick paper.

Using the procedure of the instant example, a yield of approximately 2.9% to 5.2% can be achieved. For example, 1 gram of the product can be made from 22.5-34.5 grams of unprocessed G. inflata.

### Example 2

### Extract Component Species in Various Types of Licorice

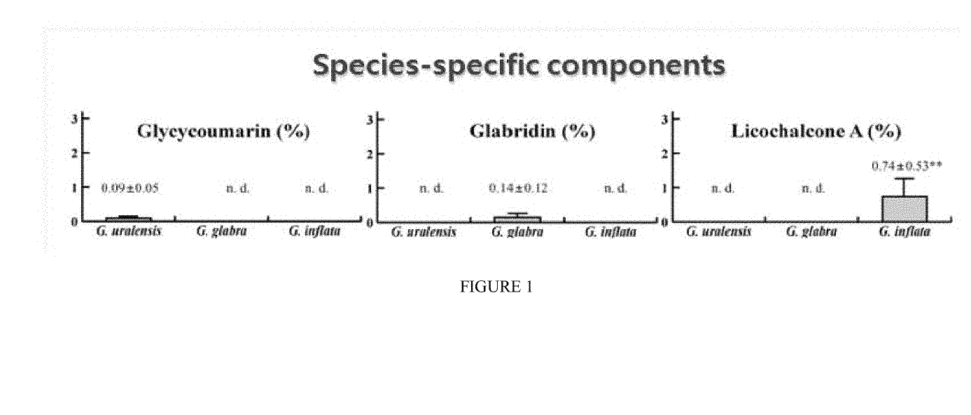

In the instant example, licorice types G. uralensis, G. glabra, and G. inflata were evaluated to determine the extract component species between the various licorice types. As shown in FIG. 1, glycycoumarin, glabridin, and licochalcone A were confirmed as the species-specific typical constituents of G. uralensis, G. glabra, and G. inflata, respectively.

G. uralensis, G. glabra, and G. inflata were purchased and component species were extracted with 95% ethanol. A comparison standard of Uralensis was obtained from Korean MFDS, and HPLC profiles were prepared for each licorice type. The HPLC profiles are shown in FIG. 2.

### Example 3

### Aqueous Extraction of Licorice Compared to Ethanol Extraction of Licorice

In the instant example, different concentrations of ethanol extraction fractions of G. inflata were prepared and compared to an aqueous extract of G. inflata. FIG. 3 shows the HPLC profile differences in aqueous extraction, 50% ethanol extraction fraction, 70% ethanol extraction fraction, and 95% ethanol extraction fraction.

### Example 4

### Evaluation of Properties from Aqueous Extracted Licorice Components Compared to Ethanol Extracted Licorice Components

In the instant example, different concentrations of ethanol extraction fractions of G. inflata were prepared and compared to an aqueous extract of G. inflata with respect to antioxidant and anti-inflammatory effects.

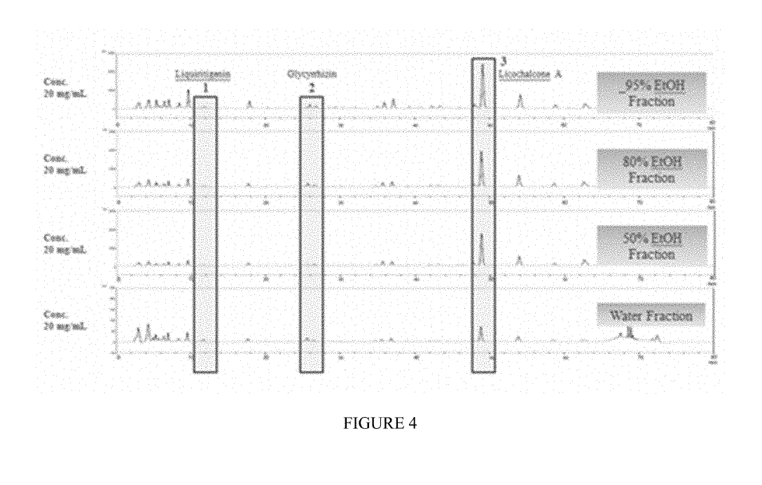

With higher concentrations of ethanol extraction, licorice extracts demonstrated improved anti-oxidative and anti-inflammatory effects. FIG. 4 and Table 1 show the highest concentration of Licochalcone A in the 95% ethanol extraction fraction.

TABLE 1

| Extraction Fraction | Content of Licochalcone A |
|---|---|
| 95% EtOH Fraction | 10.25% |
| 80% EtOH Fraction | 7.62% |
| 50% EtOH Fraction | 6.74% |
| Aqueous Fraction | 0.34% |

As shown in Table 2, the phenolic content of licorice extracts was highest in the 95% ethanol extraction fraction. The protocol involved using the Folin-Ciocalteu method based on phenolic compounds leading to reduction of a phosphotungstate-phosphomolybdate complex and generating blue reaction products (Vinson J A et al., J Agric Food Chem, 2001; 49:5315-21). The reaction mixture was reduced to 1 ml and each sample was read at 760 nm and with 30 minutes of reaction allowed to proceed in a comparison against blank water. The linear calibration curve was generated using gallic acid as the standard, allowing an absorbance range of 3 AUs. The total phenolic content values were quantified and expressed as gallic acid equivalents (GAE). The assay allowed for high reproducibility for standards and samples.

TABLE 2

| | Total phenolic contents |
|---|---|
| Extraction Fraction | (mg GAE/g of sample) |
| 95% EtOH Fraction | 291.44 |
| 80% EtOH Fraction | 199.54 |
| 50% EtOH Fraction | 171.76 |
| Aqueous Fraction | 20.02 |

The 2,2-diphenyl-1-picrylhydrazyl (DPPH) radical scavenging activity was evaluated as described (Gupta D et al., BMC Complement Altem Med, 2011; 11:13. doi: 10.1186/1472-6882-11-13) with modifications. In this assay, 0.2 mL of sample at different concentrations (1-1000 μg/mL) was added to 1.8 mL of DPPH solution (0.11 mM) in 80% ethanol. The mixture was then incubated for 30 minutes and at room temperature in the dark. The absorbance at 517 nm was then measured with ascorbic acid and butylhydroxytoluene (BHT) as reference antioxidants. A 0.2 mL of solvent extract dilution added to 1.8 mL of DPPH solution (0.11 mM) in 80% ethanol serve as the control. Two independent experiments were performed in triplicate. The percentage inhibition was calculated relative to the control using the equation, inhibition of DPPH radical (%)=(1−(Absₛₐₘₚₗₑ/Abs_{control}))×100.

As shown in Table 3, DPPH radical scavenging activity (anti-oxidant effects) was highest in the 95% ethanol extraction fraction.

TABLE 3

| | DPPH radical scavenging |
|---|---|
| Extraction Fraction | activity |
| 95% EtOH Fraction | 90.59 |
| 80% EtOH Fraction | 70.43 |
| 50% EtOH Fraction | 54.95 |
| Aqueous Fraction | 10.62 |

Furthermore, as shown in Table 4, the anti-oxidative effect of the disclosed compositions (measured by DPPH radical scavenging activity, %) was compared to ascorbic acid (an antioxidant standard) and to rebamipide (a drug approved for mucosal protection, remedying stomach ulcers and gastritis). The activity of the disclosed compositions improved by increasing the concentration of ethanol used in the extraction, and the 95% ethanol extract had the highest level of anti-oxidant activity among the extracts tested.

TABLE 4

| | Compositions of the | | |
|---|---|---|---|
| | Present Invention | Ascorbic acid | Rebamipde |
| | (50 μg/ml) | (300 μM) | (100 μg/mL) |
| DPPH radical | 90.62 ± 0.35 | 37.60 ± 0.47 | 0.93 ± 0.83 |
| scavenging | | | |
| activity, % | | | |

As shown in Table 5, reduction activity (anti-oxidant effects) was highest in the 95% ethanol extraction fraction.

TABLE 5

| | Ferric-reducing |
|---|---|
| | antioxidant power |
| Extraction Fraction | (O.D.) |
| 95% EtOH Fraction | 0.68 |
| 80% EtOH Fraction | 0.48 |
| 50% EtOH Fraction | 0.40 |
| Aqueous Fraction | 0.14 |

### Example 5

### Comparison of Licorice Extracts in LPS-Induced NO and IL-6 Production IC₅₀

In the instant example, a 95% ethanol extraction fraction showed improved STAT-3 phosphorylation compared with water extract.

% Inhibition 50 (IC50)

| | 95% | 80% | 50% | |
|---|---|---|---|---|
| | EtOH | EtOH | EtOH | Water |
| ug/ml | Extract | Extract | Extract | Extract |
| NO | 2.33 | 4.11 | 4.02 | 0.03 |
| IL-6 | 2.86 | 4.27 | 4.28 | 0.012 |

### Example 6

### Comparison of Licorice Extracts in Interleukin-6 Induced STAT-3 Phosphorylation

In the instant example, a 95% ethanol extraction fraction showed improved % Inhibition 50 (IC₅₀) values for LPS induced NO and IL-6 production compared with water extract.

| | % Inhibition | | | | |
|---|---|---|---|---|---|
| | | 95% | 80% | 50% | |
| | +Drug | EtOH | EtOH | EtOH | Water |
| ug/ml | Genestain | Extract | Extract | Extract | Extract |
| 100 uM | 96.25% | | | | |
| 1 ug/ml | — | 46.39% | 42.41% | 35.00% | 22.09% |
| 5 ug/ml | — | 97.14% | 92.41% | 88.50% | 27.07% |

### Example 6

### Evaluation of Licorice Extracts on Crohn's Disease and Ulcerative Colitis Disease Models

In the instant example, experiments were performed to evaluate a composition comprising licorice extract on Crohn's Disease and ulcerative colitis disease models. Six-week-old female BALB/c mice (Orient Bio) were and maintained under specific pathogen-free conditions at the animal facility of Bioindustry Institute (Chuncheon, Korea). To induce experimental colitis, the mice were administered 5% DSS (40,000-50,000 MW; ICN Biomedicals, Aurora, Ohio, USA) dissolved in filter-purified water (Millipore Corp, Bedford, Mass., USA). The control mice received filtered water alone.

In order to examine the therapeutic effects of the disclosed composition comprising a licorice extract component, after 3 day of applying DSS, the disclosed composition (“AD-lico”, at 25 mg and 100 mg/kg) or vehicle (5% carboxymethyl cellulose) was administrated orally for 7 days. No differences in water consumption among the groups (3.5-4.0 ml/day/mouse) were observed during the experimental period.

Assessment of Disease Activity Index Measurement: Mice were evaluated daily for colitis development by monitoring body weight, gross rectal bleeding, stool consistency, and survival. Overall disease severity was assessed using a clinical scoring system with a scale of 0-4. In brief, scoring was as follows:
0, no weight loss, no occult blood in the stools and normal stool consistency;
1, weight loss of 1-5%, no occult blood and normal stool consistency;
2, 5-10% weight loss, positive for fecal occult blood and loose stools;
3, 10-20% weight loss, positive for fecal occult blood and loose stools;
4, greater than 20% weight loss, gross rectal bleeding and diarrhea.

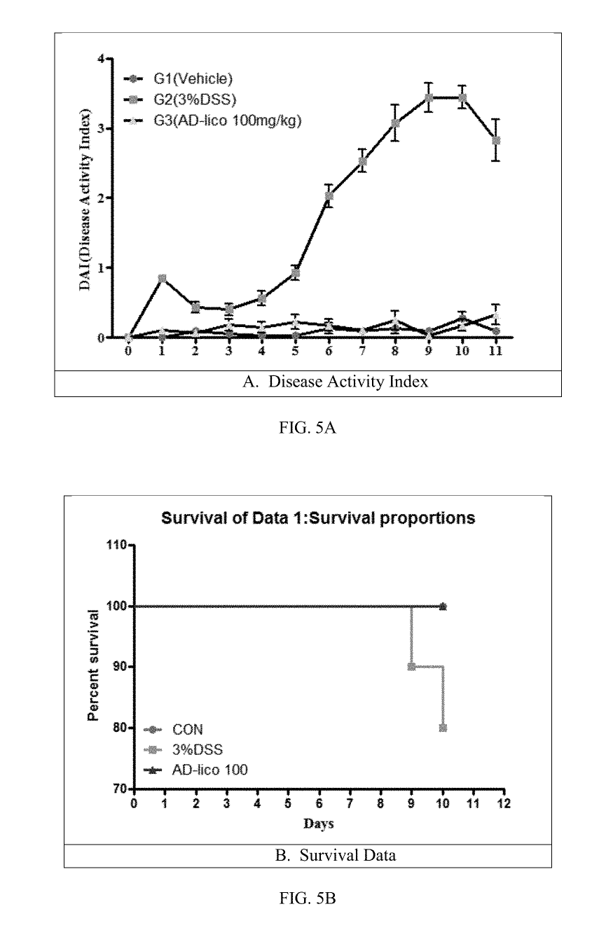

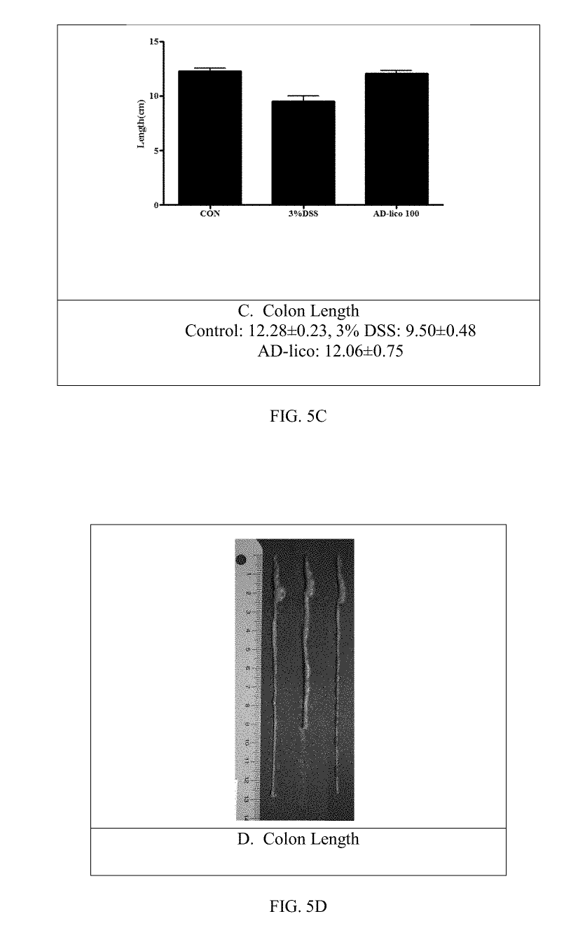

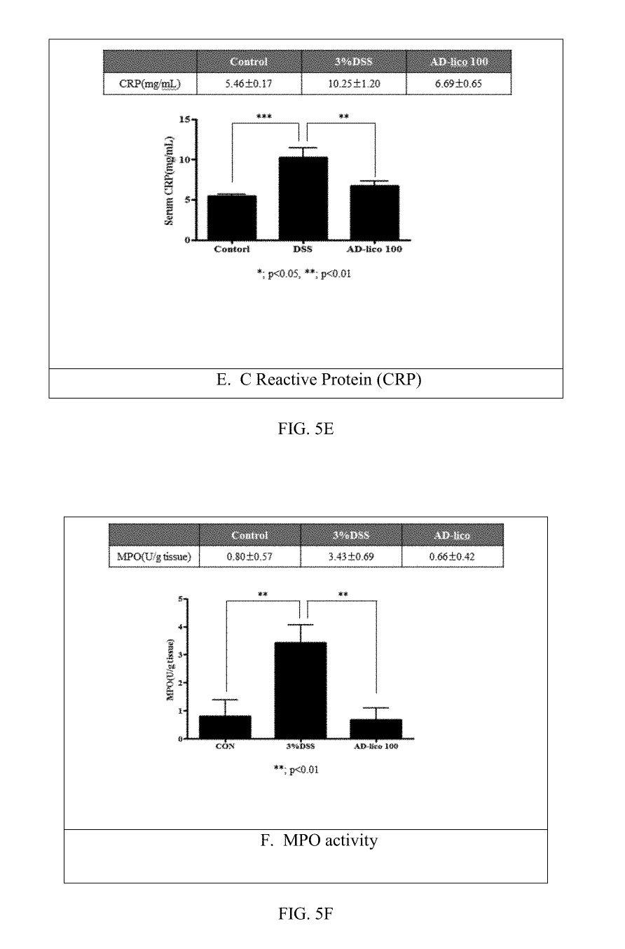

FIG. 5A compares disease activity index of a control group compared to DSS treatment and to treatment with 100 mg/kg of a composition comprising licorice extract. As shown in FIG. 5A, AD-lico demonstrated vast improvement in disease severity in the mice.

DSS Induced Survival Measurement:

Survival of the mice was evaluated every day. FIG. 5B compares survival data of a mice control group compared to DSS treatment and to treatment with 100 mg/kg of a composition comprising licorice extract. As shown in FIG. 5B, AD-lico demonstrated 100% disease survival in the mice.

Colon Length Measurement:

The DSS-induced model of colitis is associated with a significant decrease in colon length, and colon length measurement has often been used as a morphological parameter for the degree of inflammation in DSS colitis. To assess colon length, mice from each group were sacrificed after 10 days. The mice colons (50-100 mg) were rinsed with cold PBS, blotted dry, and immediately measured the length. FIGS. 5C and 5D compare colon length of a control group compared to DSS treatment and to treatment with 100 mg/kg of a composition comprising licorice extract, as well as a representation of the colons themselves. As shown in FIGS. 5C and 5D, AD-lico was effective to restore colon length to control lengths in DSS colitis mice.

C-Reactive Protein (CRP) Level Measurement:

Blood was collected 11 days after DSS activation by intracardiac puncture under Isoflurane anesthesia. Colonic samples were prepared as previously described and CRP levels were determined using enzyme-linked immunosorbent assay commercial kit (R&D Systems, Minneapolis, Minn.). FIG. 5E compares CRP measurements of a control group compared to DSS treatment and to treatment with 100 mg/kg of a composition comprising licorice extract. As shown in FIG. 5E, AD-lico was effective to reduce CRP levels to those observed in controls in DSS colitis mice.

MPO Activity Measurement:

MPO is an enzyme produced mainly by polymorphonuclear leucocytes, and it is associated with the degree of neutrophil infiltration in a given tissue. Following 10 days of DSS treatment, MPO activity became markedly increased, to a level approximately 3.3 times higher than that in the control group.

The mice colons (50-100 mg) were rinsed with cold PBS, blotted dry, and immediately free dried at −80° C. until assayed for MPO activity using the 0-dianisidine method (see, e.g., Krawisz J E et al,). To perform the assay, the tissue samples were thawed and weighed, and then suspended (10% wt/vol) in 50 mM potassium phosphate buffer (pH 6.0) containing 0.5% hexadecyltrimethylammonium bromide and homogenized. A sample of the homogenate (1 ml) was sonicated for 30 seconds, and then centrifuged at 200 g for 10 minutes at 4° C.

The reaction was started by mixing and incubating the supernatant (100 ml) at 20° C. for 10 minutes with a solution composed of 2810 ml of 50 mM potassium phosphate, 30 ml of 20 mg/ml o-dianisidine dihydrochloride, and 30 ml of 20 mM hydrogen peroxide. After 10 minutes the reaction was terminated by the addition of 30 ml of 2% sodium azide. The change in absorbance was read at 460 nm using a SpectraMax M2 Microplate Reader (Molecular Devices, Sunnyvale, Calif., USA).

MPO activity was expressed as the amount of enzyme necessary to produce a change in absorbance of 1.0 unit/min/g of tissue (wet weight). FIG. 5F compares MPO measurements of a control group compared to DSS treatment and to treatment with 100 mg/kg of a composition comprising licorice extract. As shown in FIG. 5F, the observed increase in MPO activity was significantly reduced by administration of a composition comprising licorice extract. Treatment using high concentrations (100 mg/kg) of a composition comprising licorice extract suppressed the MPO activity to basal level. Since MPO activity is considered a biochemical maker of neutrophil infiltration, this result suggests that the described composition exerts anti-inflammatory effects by reducing neutrophil infiltration into the colonic mucosa.

### Example 7

### Evaluation of Licorice Extracts on Mucosal Damage Caused by H. pylori Infection

In the instant example, compositions of the present disclosure were evaluated for their effect on mucosal damage in vivo following infection with Helicobacter pylori (H. pylori). H. pylori is the major target species of bacteria that routinely infects the stomach and has been shown to cause various stomach maladies that include peptic ulcer disease, gastritis, gastric adenocarcinoma and MALT-lymphoma. H. pylori is also difficult to treat with a relatively high treatment failure rate of 10-40% and is subject to extreme resistance to conventional antibiotics.

For the instant example, male Wistar rats weighing at 220-250 g were inoculated three times with 0.8 mL of sterile culture containing 1×10⁸ colony-forming units (CFUs) of H. pylori via gastric intubation and at 48 hours intervals. Prior to the first inoculation, the animals were fasted for 24 hours. Paraffin-embedded tissue was sectioned into 4 μm thickness slices and then stained with haematoxylin and eosin (H&E) to detect for inflammatory and epithelial changes. Infiltration of neutrophils and lymphocytes indicated mucosal damage, and the level of pathologic changes for inflammation, hyperplasia, and peptic ulceration was on a 4-point scale (0-3: 0: normal; 1: mild; 2: moderate; 3: marked) and according to the modified and updated Sydney System (Cao X et al., Cancer Sci, 2010; 98:478-483).

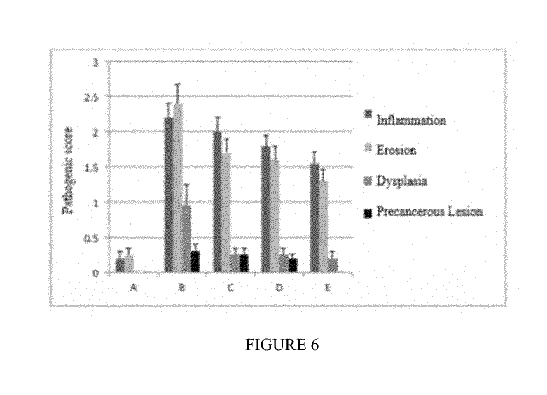

As shown in FIG. 6, a dose dependent improvement in the mucosal damage caused by H. pylori infection in rats was demonstrated. This was based on assessment of paraffin-embedded mucosal stomach samples serially sectioned and stained with H&E for histological analysis for inflammatory and/or epithelial changes. Mucosal damage was defined as infiltration by neutrophils and lymphocytes. The degree of inflammatory change, hyperplasia, and peptic ulceration was also graded according to a modified Sydney System. For all the four parameters of inflammation, erosion, dysplasia and precancerous lesions, compositions of the present disclosure (i.e., “AD-lico”) improved the evaluated parameters. The arms of the experiment were normal rats, H. pylori infected rats, and infected rats that received increasing levels of compositions of the present disclosure at 25, 50, and 100 mg/kg.

### Example 8

### Evaluation of Licorice Extracts on H. pylori Induced iNOS and COX-2 Expression

In the instant example, compositions of the present disclosure were evaluated for their effect on iNOS and COX-2 expression in gastric epithelial AGS cell cultures. Human gastric epithelial AGS cells (gastric adenocarcinoma, ATCC CRL1739) were obtained from the American Type Culture Collection (Rockville, Md., USA). Cells were cultured in RPMI-1640 medium containing 10% fetal bovine serum, 4 mM glutamine, 100 units/mL penicillin and 100 μg/mL streptomycin (GIBCO-BRL, Grand Island, N.Y., USA). The cells were seeded in 6-well cell culture plates and at the density of 3×10⁵ cells per well. Cells were cultured and used when reaching 80% confluency. Before treatment of the cells, each well was first washed with 2 ml of fresh culture medium with no antibiotics. H. pylori from a chocolate agar plate was first suspended in antibiotic-free culture medium with 10% fetal bovine serum and then added to the cultured cells at a ratio of 300:1 (bacterium/cell). This optimal ratio of cells was obtained from a previous study (Cho et al. 2010).

AGS cells treated with H. pylori were first trypsinized, and then washed. Cells were then homogenized in lysis buffer made up of Tris-HCl (pH 7.4), 1% NP-40 and a cocktail of protease inhibitors (Boehringer Mannheim, Indianapolis, Ind., USA). The protein lysate concentration for each sample was determined by the Bradford assay (Bio-Rad, Hercules, Calif., USA). Total cell extracts at 50-100 μg were separated with 7-12% SDS polyacrylamide gel electrophoresis under reducing conditions and transferred onto nitrocellulose membranes (Amersham, Arlington Heights, Ill., USA) for Western blotting. After blocking with 5% nonfat dry milk for 1 hour, the membranes were incubated with COX-2, iNOS, and actin antibodies (Santa Cruz Biotechnology, Santa Cruz, Calif.) in TBS-T containing 5% nonfat dry milk at 4° C. overnight. Following washing with TBST (Tris-buffered saline, 0.1% Tween 20), the immunoreactive proteins were visualized by using goat anti-mouse (COX-2), goat anti-rabbit (iNOS), or donkey anti-goat (actin) secondary antibodies conjugated to horseradish peroxidase and visualized by enhanced chemiluminescence (Santa Cruz Biotechnology).

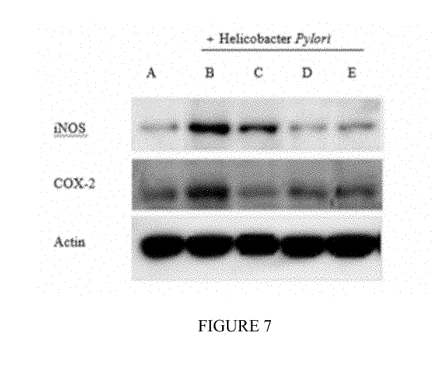

FIG. 7 shows a Western blot evaluating the indicators of inflammation, iNOS and COX-2, in human AGS gastric adenocarcinoma cells infected with H. pylori that were treated with compositions of the present disclosure (i.e., “AD-lico”). There was a significant reduction in the levels of these two markers in the extracts of the treated cells. This indicates that compositions of the present disclosure (i.e., “AD-lico”) were capable of directly acting on the stomach mucosal cells in reducing the extent of damaging inflammation.

### Example 9

### Evaluation of Licorice Extracts on Mucosal Damage Caused by Water Immersion Stress

In the instant example, compositions of the present disclosure were evaluated for their effect on mucosal damage after water immersion stress. Stress-induced gastric mucosal damage was induced in rats with a modification of a method described earlier (Takagi et al., Chem Pharm Bull (Tokyo), 1964; 12:465-72; Takagi et al., Jpn J Pharmacol, 1968; 18:9-18). The rats were restrained in special cages and were then immersed in water at 23° C. to the xiphoid level. After being exposed to 3.5 hours of water/restraint protocol, the animals were sacrificed, and their stomachs were removed, which were dissected along the greater curvature. The mucosal damage was assessed macroscopically and the number of gastric lesions was noted in each stomach of rat belonging to different experimental groups.

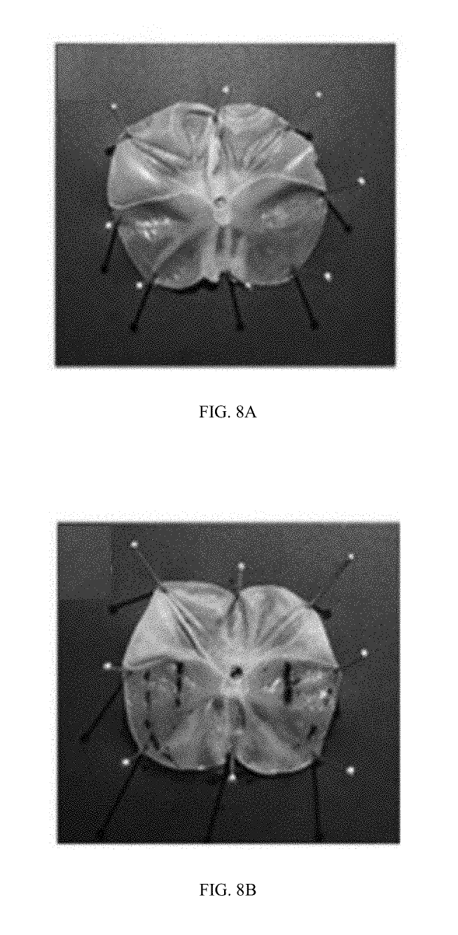

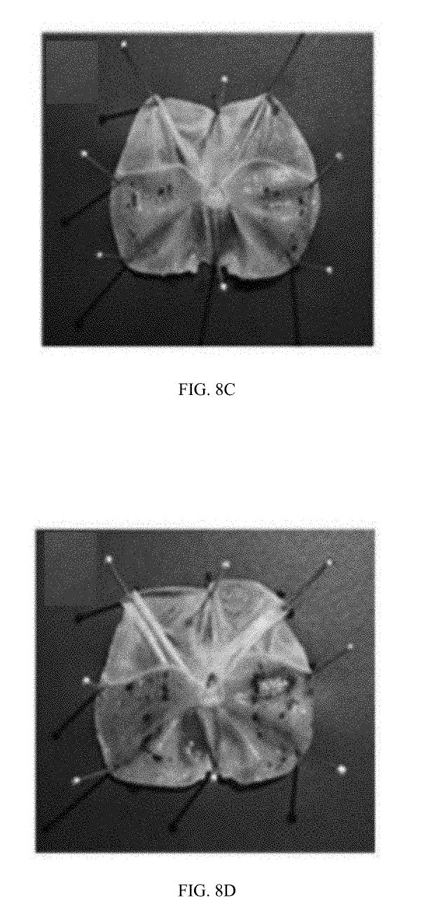

The results from the water immersion model of mucosal damage in rats are shown in FIGS. 8A-8D. The evaluation of mucosal damage caused by water immersion stress in rat stomachs are displayed in one of four groups: FIG. 8A (normal control); FIG. 8B (negative control); FIG. 8C (AD-lico, 25 mg/kg); FIG. 8D (SWG, an approved functional food in Korea, 50 mg/kg). Oral administration of compositions of the present disclosure (i.e., “AD-lico”) reduced the number of gastric lesions and damage observed visually on stomach surface of mice compared to stomach surfaces of unstressed mice as well as to stomach surfaces of mice that were stressed, but were untreated. This effect was significantly better than SWG, a functional food sold in Korea recommended to improve stomach function.

### Example 10

### Evaluation of Licorice Extracts on Gastric Emptying

In the instant example, compositions of the present disclosure were evaluated for their effect on gastric emptying. Gastric emptying (GE) was assayed according to published protocol (Ozaki A et al., Gen Pharmacol, 1999; 33:283-8). Normal male Sprague-Dawley rats at 220-250 g were fasted for 18 hours with ad libitum access to water. The rats were then given 2 ml of semi-solid meals by gavages at 60 minutes following drug administration. After 30 minutes, animals were sacrificed, and their stomachs and contents were weighed and photographed in order to determine GE.

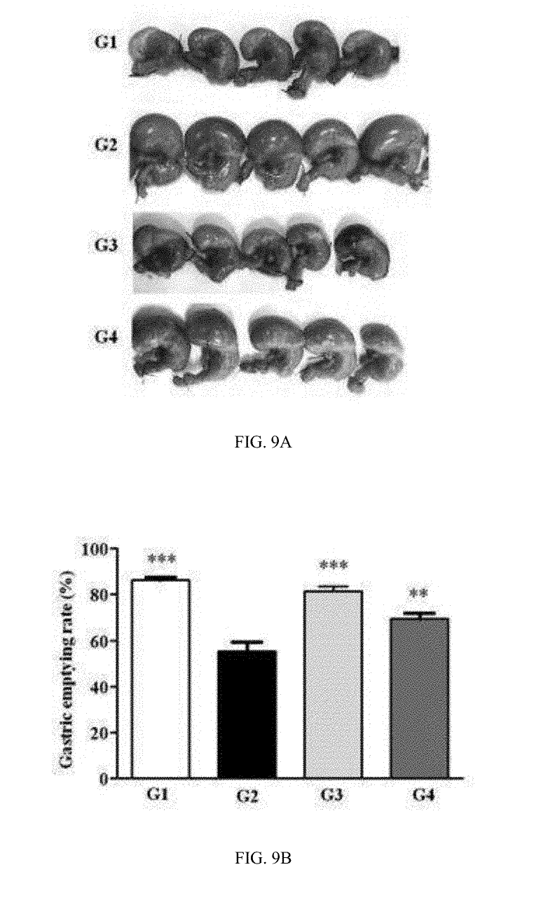

To identify potential benefits of compositions of the present disclosure (i.e., “AD-lico”) in providing relief for stomach emptying, FIGS. 9A and 9B displays the excised stomach of treated mice post feeding and the quantitation of their GE rate. FIG. 9A shows G1 (normal control); G2 (negative control); G3 (mosapride, 10 mg/kg) and G4 (AD-lico, 100 mg/kg). The rats were fasted and then given measured semi-solid meals by gavages at 60 minutes following drug administration. After 30 minutes, animals were sacrificed, and their stomachs and contents were weighed and photographed.

FIG. 9B shows quantitated gastric emptying rates of the various groups. Following drug administration, when compared with untreated controls and 30 minutes post feeding, compositions of the present disclosure (i.e., “AD-lico”)-treated group demonstrated a significantly higher GE. This improvement was comparable to mosapride, a known drug shown to improve gastroprokinetics. From this observation, compositions of the present disclosure may provide relief for patients suffering from delayed GE.

What is claimed is:

1. A medicinal composition comprising one or more extract components of a licorice.

2. The medicinal composition of claim 1, wherein the composition comprises a mixture of extract components selected from the group consisting of Licochalcone A and glycyrrhizin.

3. The medicinal composition of claim 2, wherein the extract components are present at between about 0.5% and about 6% of Licochalcone A and less than about 3% glycyrrhizin.

4. The medicinal composition of claim 1, wherein the composition comprises a mixture of extract components selected from the group consisting of Licochalcone A and echinatin.

5. The medicinal composition of claim 4, wherein the extract components are present at between about 0.5% and about 6% of Licochalcone A and between about 0.1% and about 0.5% of echinatin.

6. The medicinal composition of claim 1, wherein the composition comprises a mixture of extract components selected from the group consisting of Licochalcone A, glycyrrhizin, and echinatin.

7. The medicinal composition of claim 6, wherein the extract components are present at between about 0.5% and about 6% of Licochalcone A, less than about 3% glycyrrhizin, and between about 0.1% and about 0.5% of echinatin.

8. The medicinal composition of claim 1, wherein the extract is an ethanol extract, and wherein the ethanol extract is a 95% ethanol extract.

9. A method of treating a gastrointestinal disorder, said method comprising a step of administering a medicinal composition comprising one or more extract components of a licorice to a patient in need thereof,
wherein the gastrointestinal disorder is selected from the group consisting of gastritis, an ulcer, reflux disease, erosion, gastric discomfort, and gastric immotility.

10. The method of claim 9, wherein the composition comprises a mixture of extract components selected from the group consisting of Licochalcone A and glycyrrhizin.

11. The method of claim 10, wherein the extract components are present at between about 0.5% and about 6% of Licochalcone A and less than about 3% glycyrrhizin.

12. The method of claim 9, wherein the composition comprises a mixture of extract components selected from the group consisting of Licochalcone A and echinatin.

13. The method of claim 12, wherein the extract components are present at between about 0.5% and about 6% of Licochalcone A and between about 0.1% and about 0.5% of echinatin.

14. The method of claim 12, wherein the medicinal composition further comprises glycyrrhizin, wherein the glycyrrhizin is present at less than about 3%.

15. A method of treating an inflammatory disease, said method comprising a step of administering a medicinal composition comprising one or more extract components of a licorice to a patient in need thereof,
wherein the inflammatory disease is selected from the group consisting of an inflammatory bowel disease, ulcerative colitis, and Crohn's disease.

16. The method of claim 15, wherein the composition comprises a mixture of extract components selected from the group consisting of Licochalcone A and glycyrrhizin.

17. The method of claim 16, wherein the extract components are present at between about 0.5% and about 6% of Licochalcone A and less than about 3% glycyrrhizin.

18. The method of claim 15, wherein the composition comprises a mixture of extract components selected from the group consisting of Licochalcone A and echinatin.

19. The method of claim 18, wherein the extract components are present at between about 0.5% and about 6% of Licochalcone A and between about 0.1% and about 0.5% of echinatin.

20. The method of claim 18, wherein the medicinal composition further comprises glycyrrhizin, wherein the glycyrrhizin is present at less than about 3%.
